(19) United States
(12) Patent Application Publication
(10) Pub. No.: US 2021/0304901 A1
(43) Pub. Date: Sep. 30, 2021

(54) SYSTEMS AND METHODS FOR IDENTIFYING AND ISOLATING PERSONS SUSPECTED OF HAVING AN INFECTIOUS DISEASE

(71) Applicant: MachineSense, LLC, Baltimore, MD (US)

(72) Inventor: Biplab PAL, Ellicott City, MD (US)

(73) Assignee: MachineSense, LLC, Baltimore, MD (US)

(21) Appl. No.: 17/211,349

(22) Filed: Mar. 24, 2021

Related U.S. Application Data
(60) Provisional application No. 62/993,892, filed on Mar. 24, 2020.
(60) Provisional application No. 63/004,555, filed on Apr. 3, 2020.

(51) Int. Cl. G16H 50/80; H04W 4/029; A61B 5/01; G08B 21/18; G16H 10/60

(52) U.S. Cl. CPC G16H 50/80; H04W 4/029; G16H 10/60; G08B 21/182; A61B 5/01

(57) ABSTRACT

Systems and processes are provided for managing epidemic and pandemic disease populations by individually scanning persons in a pre-selected retention space, to identify persons having body temperatures in excess of a predetermined level. Such persons are prompted to register, and are instructed to self-quarantine at the address provided during the registration process. The location of the registered person subsequently is tracked to ensure that the person remains at the registered location during the quarantine period. The registration information can be provided to health and law enforcement authorities to help control the spread of the disease population.

### CROSS-REFERENCE TO RELATED APPLICATIONS

This application claims the benefit under 35 U.S.C. § 119(e) of U.S. application no. 62/993,892, filed Mar. 24, 2020, and U.S. application no. 63/004,555, filed Apr. 3, 2020. The contents of each of these applications are incorporated by reference herein in their entirety.

### BACKGROUND OF THE INVENTION

Highly-contagious diseases that reach epidemic and pandemic proportions, such as COVID-19, the 1918 Spanish Flu, SARS, EBOLA, etc., present a grave threat to public health and safety. Slowing the spread of such diseases generally requires identifying infected individuals, and isolating such individuals from the rest of the population until they no longer have the potential to infect others.

The mobility of people in a modern society presents a significant challenge to identifying and isolating individuals suffering from an infectious disease. For example, as a result of modern air travel, hundreds of people from a single flight can enter a local a population on a virtually instantaneous basis. As another example, modern shopping malls typically accommodate thousands of people congregating in close proximity in an indoor area. If such large, mobile groups of people cannot quickly and reliably be screened for infectious diseases in the midst of an epidemic or a pandemic, the spread of such diseases can be very difficult if not impossible to control.

### SUMMARY OF THE INVENTION

In one aspect of the disclosed technology, a method is provided for managing epidemic and pandemic disease populations by individually scanning persons in a pre-defined group in a pre-selected retention space, to identify those in a group having body temperatures in excess of a predetermined level. Each identification of a person having body temperature in excess of the predetermined level defines an event.

In another aspect of the disclosed technology, the method elicits facial recognition from each of the identified persons.

In another aspect of the disclosed technology, the method requires each identified person to download an identified application onto their personal electronic device, and to use the application to register information concerning themselves, including, for example, their name, contact information, age, body temperature, address for self-quarantine, and recent travel and visitation history, as a pre-condition to release from the retention space.

In another aspect of the disclosed technology, the application notes the event and the time occurrence thereof.

In another aspect of the disclosed technology, the registered information for each event is transferred to a governmental authority via the internet or other suitable transmission means.

In another aspect of the disclosed technology, a geofencing quarantine perimeter is defined for each registered individual, and the individual is periodically or continually monitored to determine whether the individual remains within the quarantine perimeter the duration of the quarantine period.

In another aspect of the disclosed technology, the registered information for each event is collected, the number of events within a particular timeframe are counted, and the percentage of persons in the pre-defined group having body temperatures in excess of the predetermined level during the timeframe are computed.

In another aspect of the disclosed technology, a system includes infrared (IR) sensors arranged in a constellation, and an ultrasonic sensor. The IR sensors and the ultrasound sensor are arranged so that the sensors can sense human body temperature from an allowable distance so that skin temperature is detected with a relatively low number of false positive and false negative readings, i.e., detecting a high temperature when body temperature is low and vice versa.

In another aspect of the disclosed technology, measured skin temperature is correlated with core body temperature to account for differences between the two. Such differences can be substantial during winter conditions, i.e., with outdoor temperatures below about 40° F., and summer conditions, i.e., outdoor temperatures above about 100° F., and unless accounted for, can lead to possible false negative and false positive indications for elevated body temperature. The computation can be performed by an edge computation engine such an edge-cloud server.

In another aspect of the disclosed technology, a temperature scan of an individual's head is obtained while the individual is moving under an arch equipped with one or more IR sensors aimed towards the head.

In another aspect of the disclosed technology, a temperature sensor includes two IR sensors spaced apart by about four inches to about 12 inches, with their respective sensing surfaces facing each other. To obtain a temperature scan, an individual places his or her hand between two sensors without contacting either sensor. As a result of the sensor arrangement, at least one of the IR sensors will reliably detect the skin temperature of the hand regardless of the orientation of the hand.

In another aspect of the disclosed technology, a ring-shaped IR sensor is used to as part of a doorbell. Doorbells typically have to be pressed, and therefore present a potential source of disease spread at the entrance of a home. With the IR-sensing doorbell, a person desiring to activate the doorbell places the end of a finger inside the ring-shaped IR sensor. The ring-shaped IR sensor has an inside diameter that is substantially greater than the maximum height and width of a typical finger, so that finger can enter the ring without touching any adjacent surfaces. The IR sensor detects the presence of the finger based on its heat signature, and generates an output that results in activation of the doorbell. The doorbell is configured so that the sound emitted by the doorbell changes when the measured temperature of the user's finger is above a predetermined threshold, providing the homeowner with an indication that the person ringing the doorbell may be ill.

In another aspect of the disclosed technology, IR sensors can be retrofitted to existing door frames, and/or to rack frames in retail spaces, so that individuals coming close to the sensor can undergo a temperature scan, thereby providing an estimate of the number of people visiting the space, and whether those people do or do not have temperatures indicating an illness.

In another aspect of the disclosed technology, one of the IR sensors scans a standardized cold body using thermoelectric effect(s), and the remaining IR sensors scan body radiation temperature. Sensing cold body temperature using dye normally slows down the IR scanning speed, but when a calibrated external cold source is used as a standard, the IR scanning speed is increased.

In another aspect of the disclosed technology, the temperature data is stored in a cloud.

In another aspect of the disclosed technology, persons detected with high body temperature are tasked to download a geofencing application, so that authorities can be provided with an indication that individuals with high temperatures have been quarantined at home.

In another aspect of the disclosed technology, a system for identifying and isolating persons suspected of having an infectious disease includes a gateway configured to define a space for the passage of a person therethrough; a temperature sensor configured to, during operation, generate a temperature reading of the person as the person passes through the space; and a computing system communicatively coupled to the temperature sensor. The computing system is configured to, during operation, determine whether the temperature reading exceeds a limit, and if so, prompt the person to input information into the computing system.

In another aspect of the disclosed technology, the system also includes a video camera communicatively coupled to the computing system and configured to, during operation, generate an image of the person as the person passes through the space.

In another aspect of the disclosed technology, the system also includes an alarm communicatively coupled to the computing system and configured to, during operation, generate a visual and/or an audible alert; wherein the computing device is further configured to, during operation, activate the alarm when the temperature reading exceeds the limit.

In another aspect of the disclosed technology at least one of the temperature sensor, the video camera, and the alarm are mounted on the gateway.

In another aspect of the disclosed technology, the temperature sensor is an infrared sensor.

In another aspect of the disclosed technology, the information includes registration information relating to at least one of: an identify of the person; a residence of the person; a travel history of the person during a predetermined time period; and a listing of public places visited by the person during a predetermined time period.

In another aspect of the disclosed technology, the computing system is further configured to, during operation, monitor a location of the person.

In another aspect of the disclosed technology, the computing system is further configured to, during operation, determine whether the person is located at a specific location.

In another aspect of the disclosed technology, the computing system is further configured to, during operation, generate and send a notification upon determining that the person is not located at the specific location.

In another aspect of the disclosed technology, the specific location is a residence of the person.

In another aspect of the disclosed technology, the computing system is further configured to, during operation, send the notification to the person and/or to a governmental authority.

In another aspect of the disclosed technology, the computing system is further configured to, during operation, prompt the person to input the information into the computing system by way of a smart phone.

In another aspect of the disclosed technology, the computing system is further configured to, during operation, determine whether the person is located at the specific location based on location information generated by a smart phone.

In another aspect of the disclosed technology, the system further includes an ultrasonic sensor communicatively coupled to the computing device and configured to, during operation, generate an output representing a distance between the ultrasonic sensor and the person as the person passes through the space.

In another aspect of the disclosed technology, the computing system is further configured to, during operation, determine a location of the person in relation to the gateway based on the output of the ultrasonic sensor; and, if the location lies outside of a particular range, to ignore the temperature reading.

In another aspect of the disclosed technology, the computing system is further configured to, during operation, calculate a core body temperature of the individual based on the temperature reading, an ambient temperature measured at or near the gateway, and a relationship between ambient temperature, skin temperature, and core body temperature.

In another aspect of the disclosed technology, the computing system is further configured to, during operation, determine whether the core body temperature exceeds the limit, and if so, prompt the person to input the information into the computing system.

In another aspect of the disclosed technology, the computing system is further configured to, during operation, interpret a core body temperature that exceeds the limit as an indication that the person has a fever.

In another aspect of the disclosed technology, the relationship between ambient temperature, skin temperature, and core body temperature accounts for differences between skin temperature and core body temperature based on the ambient temperature.

In another aspect of the disclosed technology, the relationship between ambient temperature, skin temperature, and core body temperature is determined using a training data set.

In another aspect of the disclosed technology, a process for identifying and isolating persons suspected of having an infectious disease includes directing a person through a space; measuring a temperature of the person as the person passes through the space; determining whether the temperature exceeds a limit, and if so, prompting the person to provide information regarding the identity and residence of the person; and monitoring the location of the person.

In another aspect of the disclosed technology, directing a person through a space comprises directing the person through a gateway that defines the space.

In another aspect of the disclosed technology, the process further includes obtaining a visual image of the person as the person passes through the space.

In another aspect of the disclosed technology, the process further includes generating a visual and/or an audible alert when the temperature reading exceeds the limit.

In another aspect of the disclosed technology, the process further includes generating an alert if the temperature reading exceeds the limit.

In another aspect of the disclosed technology, the process further includes monitoring a location of the person.

In another aspect of the disclosed technology, the process further includes determining whether the person is located at a specific location.

In another aspect of the disclosed technology, the process further includes generating and sending a notification upon determining that the person is not located at the specific location.

In another aspect of the disclosed technology, determining whether the person is located at a specific location includes determining whether the person is located at a residence of the person.

In another aspect of the disclosed technology, the process further includes sending the notification to the person and/or to a governmental authority.

In another aspect of the disclosed technology, the process further includes prompting the person to input information into the computing system by way of a smart phone.

In another aspect of the disclosed technology, the process further includes determining whether the person is located at the specific location based on location information generated by a smart phone.

In another aspect of the disclosed technology, the process further includes determining a position of the person within the space; and, if the position of the person lies outside of a particular range, ignoring the measured temperature.

In another aspect of the disclosed technology, determining a position of the person within the space includes determining the position of the person using an ultrasonic sensor.

In another aspect of the disclosed technology, the process further includes calculating a core body temperature of the person based on the measured temperature of the person, an ambient temperature measured at or near the gateway, and a relationship between ambient temperature, skin temperature, and core body temperature.

In another aspect of the disclosed technology, the process further includes determining whether the core body temperature exceeds the limit, and if so, prompting the person to provide the information regarding the identity and residence of the person.

### DESCRIPTION OF THE DRAWINGS

The following drawings are illustrative of particular embodiments of the present disclosure and do not limit the scope of the present disclosure. The drawings are not to scale and are intended for use in conjunction with the explanations provided herein. Embodiments of the present disclosure will hereinafter be described in conjunction with the appended drawings.

FIG. 1 is a diagrammatic illustration of an embodiment of a system for identifying and isolating persons suspected of having an infectious disease.

FIG. 2 is a diagrammatic illustration of the system shown in FIG. 1.

FIG. 3 is a diagrammatic illustration of a computing device of the system shown in FIGS. 1 and 2.

FIG. 4 is a flow chart depicting an example of a process for identifying and isolating persons suspected of having an infectious disease.

FIG. 5 is a diagrammatical illustration of the system shown in FIGS. 1-3 interfacing with a police station, a self-quarantining individual, and a security control room.

FIG. 6 depicts an example of a timeline graph generated by the system shown in FIGS. 1-3 and 5, displaying temperatures of the individuals as measured by the system and the times at which the respective temperature measurements were obtained.

FIG. 7 depicts an example of a tabular summary generated by the system shown in FIGS. 1-3 and 5, listing an event number, the measured temperature of the individual causing the event, the time and location of the event, whether the individual has been registered as discussed below, and a total event count.

FIGS. 8A-8C depict examples of interactive registration pages generated by the system shown in FIGS. 1-3 and 5 and displayed on a smart phone.

FIG. 9 is an example of a format for e-mails that can be used to notify public health officials of persons identified as potentially having an infectious disease.

FIG. 10 is a diagrammatic illustration of a temperature sensor capable of use with the system shown in FIGS. 1-3 and 5.

### DESCRIPTION OF THE INVENTION

The following detailed description is merely exemplary in nature and is not intended to limit the described embodiments of the invention or uses of the described embodiments. As used herein, the words “exemplary” and “illustrative” mean “serving as an example, instance, or for illustration.” Any implementation or embodiment or abstract disclosed herein as being “exemplary” or “illustrative” is not necessarily to be construed as preferred or advantageous over other implementations, aspects, or embodiments. All of the implementations or embodiments described in the detailed description are exemplary implementations and embodiments provided to enable persons of skill in the art to make and to use the implementations and embodiments as disclosed below, to otherwise practice the invention, and are not intended to limit the scope of the invention, which is defined by the claims.

Furthermore, by this disclosure, there is no intention on the part of the Applicant to be bound by any express or implied theory presented in the preceding materials, including but not limited to the summary of the invention or the description of the prior art, or in the following detailed description of the invention. It is to be understood that the specific implementations, devices, processes, aspects, and the like illustrated in the attached drawings and described in the following portion of the application, usually referred to as the “specification,” are simply exemplary embodiments of the inventive concepts defined in the claims. Accordingly, specific dimensions and other physical characteristics relating to the embodiments disclosed herein are not to be considered as limiting as respecting the invention unless the claims or the specification expressly state otherwise.

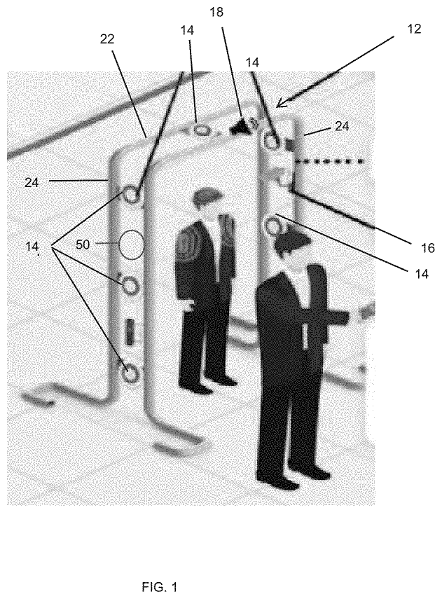

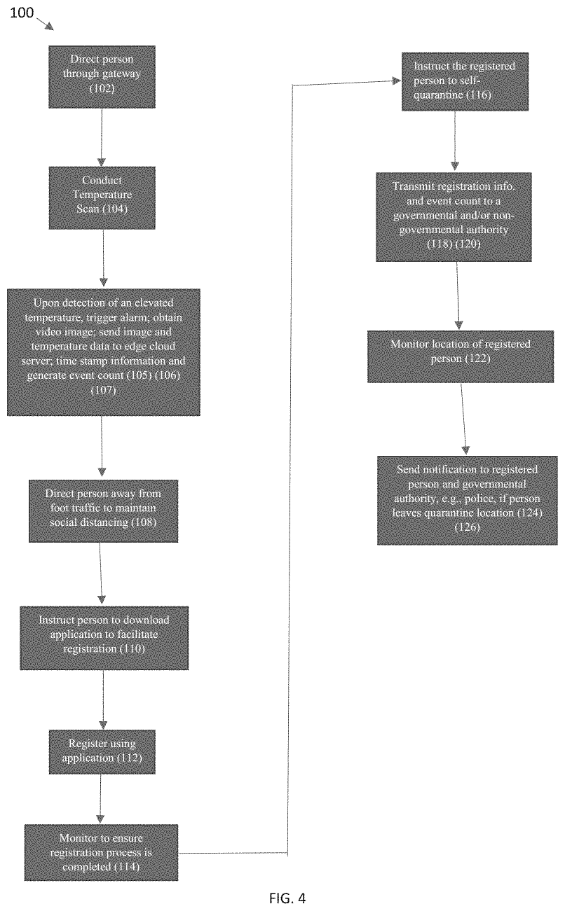

FIGS. 1-3 and 5 depict an embodiment of a system 10 for reducing the spread of infectious diseases in a population of humans by identifying and isolating persons suspected of having such diseases. FIG. 4 depicts an exemplary process 100 that can be performed using the system 10 and variants thereof. The system 10 and method 100 can be used to screen individuals entering or exiting a particular space for elevated body temperature, i.e., fever, which can indicate that the individual is afflicted with an infectious disease or other illness.

Once an individual with an elevated body temperature is identified, the system 10 and method 100 facilitate registration of the individual in a centralized data base, so that the individual can be monitored while he or she self-quarantines for an appropriate period to reduce the potential for the individual to infect others. Also, some or all of the registration information can be forwarded to appropriate governmental authorities and/or other organizations, where the information can be used to track or monitor the spread of infectious diseases.

In the illustrative embodiment disclosed herein, the system 10 is used at the exit of passenger gate in an airport, to screen passengers arriving on incoming flights. This particular application is described for exemplary purposes only. The system 10, and variants thereof, can be used to screen individuals in other settings such as train stations, bus stations, shopping malls, grocery and other retail stores, office buildings, industrial operations, and other areas having discrete entrance or exit points.

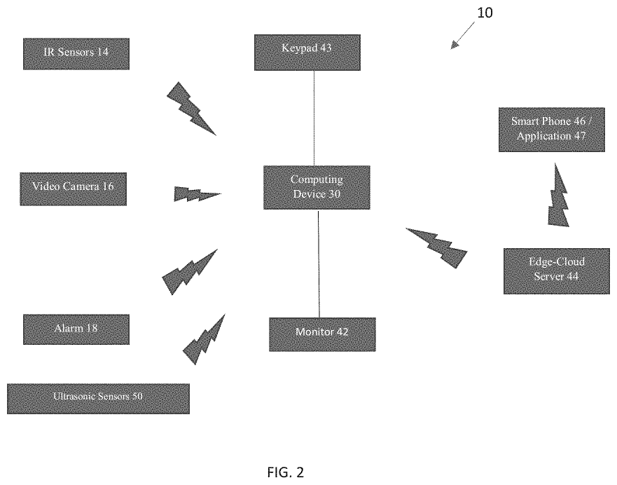

Referring to FIGS. 1 and 2, the system 10 comprises a gateway 12, one or more infrared sensors 14, a video camera 16, and an alarm 18. The infrared sensors 14, video camera 16, and alarm 18 are mounted on, or proximate the gateway 12. The system 10 also includes a computing device 30. The computing device 30 is mounted on, or proximate the gateway 12, and is communicatively coupled to the infrared sensor 14, video camera 16, and the alarm 18 by a suitable wired or wireless means.

The gateway 12 is shaped as an inverted U, and has a first side portion 20, a second side portion 22, and a transverse or top portion 24 that adjoins the first and second side portions 20, 22. The first and second side portions 20, 22 and the top portion 24 define an opening 26, and are sized so that an adult human of above-average height and weight can pass through the opening 26 comfortably, without coming into contact with the gateway 12.

During operation of the system 10, individuals being screened are directed through the opening 26. The IR sensors 14 and the video camera 16 are positioned so that the IR sensor 14 and the video camera 16 have a direct line of sight to the individual passing through the gateway 12, as shown in FIG. 1. The gateway 12 helps to direct the individual to a position at which the IR sensors 14 can obtain a temperature scan of the individual, and the video camera 16 can obtain a video image of the individual.

The gateway 12 is depicted as being shaped as an inverted U for illustrative purposes only. The gateway 12 can have other shapes in alternative embodiments. For example, the gateway can be arch-shaped, and the IR sensors 14 and the video camera 16 can be arranged to obtain a thermal scan and a video image as the individual's head passes through the arch.

In other alternative embodiments, the IR sensors 14 and the video camera 16 can be mounted on an existing ceiling, door frame or other architectural feature. In further alternative embodiments, the IR sensors 14 and the video camera 16 can be mounted on a clothing rack, a shelf, or other structure in a retail space, so that individuals coming into proximity with the IR sensors 14 can undergo a temperature scan. Thus, an interested party, such as the management of a retail store, can be provided with an estimate of the number of people visiting a space of interest, and with an indication whether one or more of the individuals has an elevated body temperature indicating an illness.

The IR sensors 14 performs a temperature scan of the individual passing through the gateway 12, and provide an output to the computing device 30 indicating of the skin temperature of the individual. The IR sensors 14 can be configured to perform high-speed scanning, e.g., 1000 temperature samples per second, thereby allowing the maximum saturated temperature value to be obtained more effectively, and in less time than otherwise would be possible. The IR sensors 14 can include firmware that recognizes the forehead or the area around the eyes of the scanned individual through IR imaging, and registers the temperature of that particular area. In some possible embodiments, the system 10 can use an infrared image of the face of the person passing through the gateway 12, as generated by the IR sensors 14; and an optical image of the person's face as generated by the video camera 16, to identify the skin temperature at each position on the person's face, provided the infrared and video images have approximately the same resolution. Other techniques for identifying the skin temperature in a particular area on the scanned individual can be used in the alternative.

The gateway 12 is depicted as being equipped with seven IR sensors 14. Alternative embodiments can include more, or less than seven IR sensors 14.

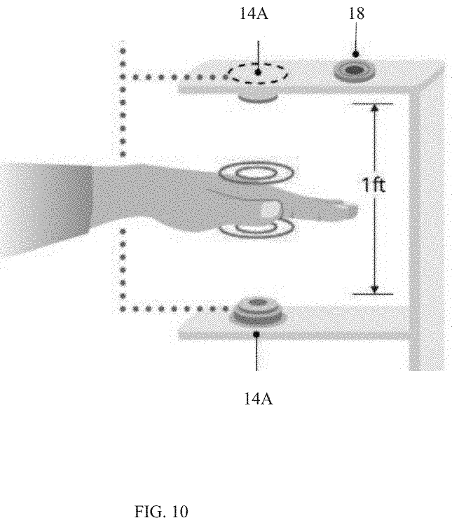

Referring to FIG. 10, alternative embodiments can include two IR sensors 14a that are spaced apart by about four inches to about 12 inches, with their respective sensing surfaces facing each other. To obtain a temperature scan, an individual places his or her hand between the two sensors 14a without contacting either sensor 14a. Due to the noted arrangement of the IR sensors 14a, at least one of the IR sensors 14a will reliably detect the skin temperature of the hand regardless of the orientation of the hand in relation of the IR sensors 14a.

In another alternative embodiment, the system includes an additional temperature sensor configured to thermally scan a standardized cold body using a thermoelectric technique, such as the Peltier effect, to account for blackbody radiation. Such use of a calibrated external cold source as a standard can help increase the overall scanning speed in relation to a normal IR sensor by eliminating the additional scanning time associated with comparing the measured temperature readings with the temperature measurements of the cold dye.

The video camera 16 captures an electronic image of the face of an individual passing through the gateway 12. The video camera 16 includes firmware that generates a digitized representation of the individual suitable for use by facial recognition software, and transmits the image to the computing device 30.

The alarm 18 can be a visual alarm such as a bright flashing light; and/or an audible alarm such as a steady or undulating siren or buzzer. The alarm 18 is activated and deactivated in response to inputs generated by the computing device 30.

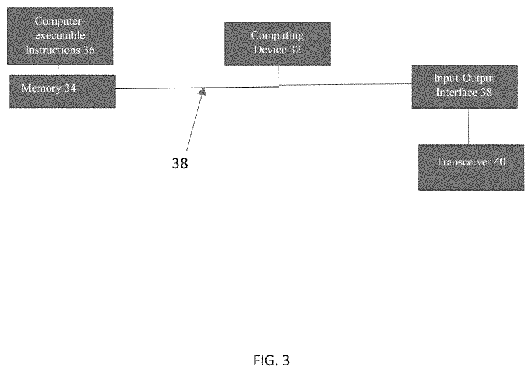

The computing device 30 can be, for example, a mini-computer. The computing device 30 can have other forms in alternative embodiments. Referring the FIG. 3, the computing device 30 comprises a processor 32. The processor 32 can be, for example, a microprocessor. The computing device 30 also includes a memory 34, such as a random access memory, communicatively coupled to the processor 32; and computer executable instructions 36 stored on the memory 34. The processor 32 is configured so that the processor 32, upon executing the computer executable instructions 36, carries out the logical operations discussed below.

The computing device 30 also comprises an internal bus 38 that facilitates communications between the various components of the computing device 30; and an input-output interface 38 communicatively coupled to the processor 32. The computing device 30 further includes a transceiver 40 communicatively coupled to the input-output interface 38 and configured to facilitate wireless communications to and from the computing device 30.

Specific details of the computing device 30 are presented for illustrative purposes only. The computing device 30 can have other configurations in alternative embodiments.

The system 10 further includes provisions to permit an authorized user to interface with the computing device 30. For example, the system 10 can include a monitor 42 and a keypad 43 communicatively coupled to the computing device 30 as depicted in FIG. 2.

. The system 10 also includes an edge-cloud server 44, shown in FIG. 2. The edge cloud server 44 can be positioned at a suitable location on or off-site from the premises at which the gateway 12 is located. The edge-could server 44 can communicate with the computing device 30 via a suitable means such as, without limitation, Wi-Fi, a cellular network, a local area network, a wide area network, or a wired connection. The edge-could server 44 is configured to perform the data-processing, data storage, and other operations discussed below. The edge cloud server 44 and the computing device 30 together form a computing system.

The edge-cloud server 44 is depicted as being communicatively coupled to one computing device 30 associated with one gateway 12. In alternative embodiments, the edge cloud server 44 can be communicatively coupled to, and can process data from multiple computing devices 30 each associated with a different gateway 12. For example, one edge cloud server 44 can be positioned at or near an airport to process data from multiple computing devices 30 each associated with a respective gateway 12 located at a particular boarding gate within the airport.

The system 10 can be used to screen individuals entering or exiting a particular area for an elevated body temperature indicating that an individual has a fever and possibly is afflicted with an infectious disease. The gateway 12 is positioned at or proximate the entrance or exit of the area. Individuals entering or exiting the area are directed through the opening 26. In the illustrative embodiment disclosed herein, the gateway 12 can be positioned at or near the exit from the passenger boarding gate at an airport, to screen arriving passengers for diseases such as Covid-19. Passengers exiting an aircraft arriving at that particular gate enter a single jetway immediately upon exiting the aircraft, and the jetway directs the arriving passengers to the boarding gate. Thus, all of the passengers exiting the aircraft in a normal manner are funneled through the opening 26 of the gateway 12 as they depart the aircraft, helping to ensure adequate screening of the arriving passengers (activity 102 of FIG. 4).

The IR sensors 14 constantly scan the opening 26 of the gateway 12, and obtain a temperature scan of an individual passing through the opening 26 of the gateway 12 (activity 104). The temperature readings are transmitted to the computing device 30. The computer-executable instructions 36 of the computing device 30 are configured to, upon execution by the processor 32, recognize a maximum temperature above a predetermined threshold, e.g., 100.5° F., as an indication that the scanned individual has an elevated body temperature indicating a fever and possible infection with a disease. The temperature threshold can be adjusted by an authorized user by way of the monitor 42 and the keypad 43. For example, the temperature threshold can be set at a level recommended by local or central health authorities based on a particular public health concern at the time. In alternative embodiments, the temperature-monitoring function can be performed by the edge-cloud server 44 instead of the computing device 30.

In alternative embodiments, the computer-executable instructions 36 are further configured to, upon execution by the processor 32, maintain an average of the temperature readings over a predetermined time period, e.g., over the previous hour; and to characterize as an elevated temperature any temperature reading that exceed the running average by a predetermined amount. This technique can help to account for variations in the average body temperatures of the screened individuals resulting from factors such as unusually high or low ambient temperatures.

The skin temperature measured by the IR sensors 14 can be correlated with the individual's core body temperature, to account for differences between the two. Such differences can be substantial during winter conditions, i.e., with outdoor temperatures below about 40° F., and summer conditions, i.e., with outdoor temperatures above about 100° F. Because fever is manifested by a rise in the core body temperature, the core body temperature can be used by the edge-cloud server to determine whether the individual passing through the gateway 12 has a fever indicating a possible infectious disease. The use of core body temperature thus can help reduce false positive and false negative indications of elevated temperature in persons passing through the gateway 12.

In practice, a correlation formula can be developed through the use of a training dataset that includes skin temperatures measured under different ambient temperatures, and corresponding core body temperatures. During operation of the system 10, the process of determining core body temperature based on the skin-temperature readings from the IR sensors 14 can be performed by the edge-cloud server 44, to help minimize the computational load on the computing device 30. In particular, the edge-cloud server 44 receives the ambient temperature measurement from a temperature sensor (not shown) located on or near the gateway 12. The edge-cloud server 44 applies the pre-determined correlation to the measured ambient temperature, and to the skin temperature as measured by the IR sensors 14, to yield a core body temperature for the individual passing through the gateway 12. A core body temperature above the pre-determined threshold is interpreted by the edge-cloud server 44 and the computing device 30 as an elevated temperature indicating a possible infectious disease in the individual passing through the gateway 12.

The system 10 further includes one or more ultrasonic sensors 50 for measuring the distance between the IR sensors 14 and the individual passing through the gateway 12. The ultrasonic sensors 50 are communicatively coupled to the computing device 30, and are depicted in FIGS. 1 and 2 (only one of the ultrasonic sensors 50 is depicted in FIG. 2, for clarity of illustration). Each ultrasonic sensor 50 is configured to transmit ultrasonic energy toward the individual, and to generate an output indicating the distance between the ultrasonic sensor 50 and the individual based on the reflection, or echo, of the ultrasonic energy from the individual. The computer-executable instructions 36 of the computing device 30 are configured to, upon execution by the processor 32, use the distance readings to determine the location of the individual within the opening 26 defined by the gateway 12. The computer-executable instructions 36 are further configured to recognize a location outside of a particular range of locations as an indication that the temperature measurements from the IR sensors 14 may not be sufficiently accurate. In particular, the optimal signal to noise ratio in the temperature measurement provided by the IR sensor is achieved at a moderate sensing distance. If an individual passing through the gateway 12 is too close to a particular IR sensor 14, the calibration for that IR sensor 14 may not be valid at that distance. If the individual is too far from the IR sensor 14, the resulting temperature measurement may be excessively influenced by the ambient temperature. Thus, the computer-executable instructions 36 can be configured to ignore a temperature reading from a particular IR sensor 14 when the readings from the ultrasonic sensors 50 indicate that individual passing through the gateway 12 is too close to, or too far from the IR sensors 14 for the IR sensors 14 to provide reliable temperature readings.

This technique thus can help to reduce false positive and false negative indication of elevated temperature in persons passing through the gateway 12.

The detection of an elevated temperature, i.e., a measured body temperature above the predetermined threshold, triggers an event count, and registration of the individual having the elevated temperature. More particularly, upon the detection of an elevated temperature reading for a particular individual, the computing device 30 triggers the video camera 16 to obtain an image of the individual (activity 105). The measured temperature and the image are transmitted to the edge-cloud server 44, which records and timestamps the information, and generates an event count (activity 106).

In applications where more than one gateway 12 is being serviced by the edge-cloud server 44, a unique identifier and/or the location of the particular gateway 12 generating the information also are recorded. Also, the computing device 30 is configured to trigger the alarm 18 to alert the individual passing through the gateway 12 that he or she has been identified as having an elevated temperature, and to alert any security personnel who may be monitoring the area (107).

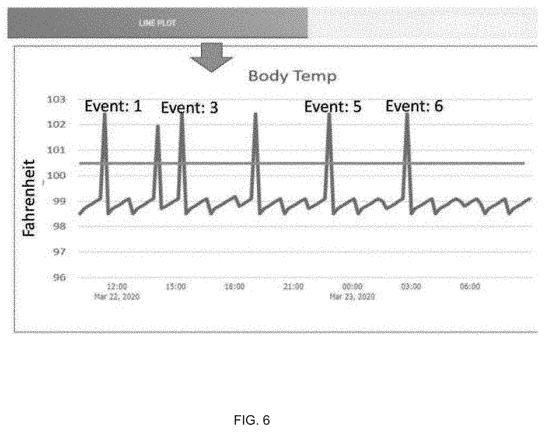

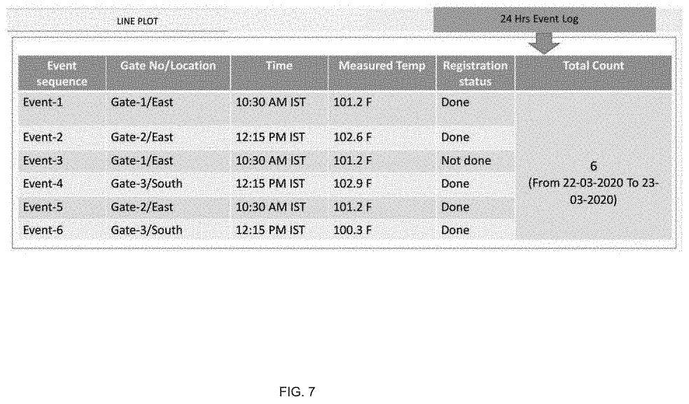

The edge-cloud server 44 can generate and maintain a timeline graph shown in FIG. 6, in which the measured temperatures of the individuals passing through the gateway 12 are presented on vertical axis, and the respective times at which the individuals passed through the gateway 12 are presented on the horizontal axis. Instances where the measured temperature of a particular individual exceeded the predetermined threshold are flagged on the graph as an event. Also, the edge-cloud server 44 can generate and maintain a tabular summary of the events as shown in FIG. 7, in which, for example, an event number, the measured temperature of the individual causing the event, the time and location of the event, whether the individual has been registered as discussed below, and a total event count are presented. The timeline graph and tabular summary can be provided to interested organizations such as local, regional, and/or national public health authorities.

Upon being identified as having an elevated body temperature, the individual can be directed to an area outside of the flow of foot traffic, to help maintain a proper social distance between the individual and others and thereby reduce the potential for the individual to infect others (activity 108). If desired in a particular application, a security guard, medical professional, or other attendant can be stationed at the exit of the gateway 12 to re-check the individual's temperature using a hand-held thermometer or other suitable means, to help minimize any impact from false-positive elevated temperature readings from the IR sensor 14.

Alternative embodiments of the gateway 12 can be equipped with a movable physical barrier (not shown) that selectively prevents passage through the gateway 12 by individuals found to have elevated body temperature. The barrier can be communicatively coupled to the computing device 30, and can be moved to its closed position immediately upon detection of an elevated body temperature in the individual passing through the gateway 12, thereby blocking passage of the individual through the gateway 12. Alternatively, the barrier normally can reside in its closed position, and can be moved to the open position immediately upon a determination that the individual passing through the gateway 12 does not have an elevated body temperature, so that the individual can pass through the gateway 12.

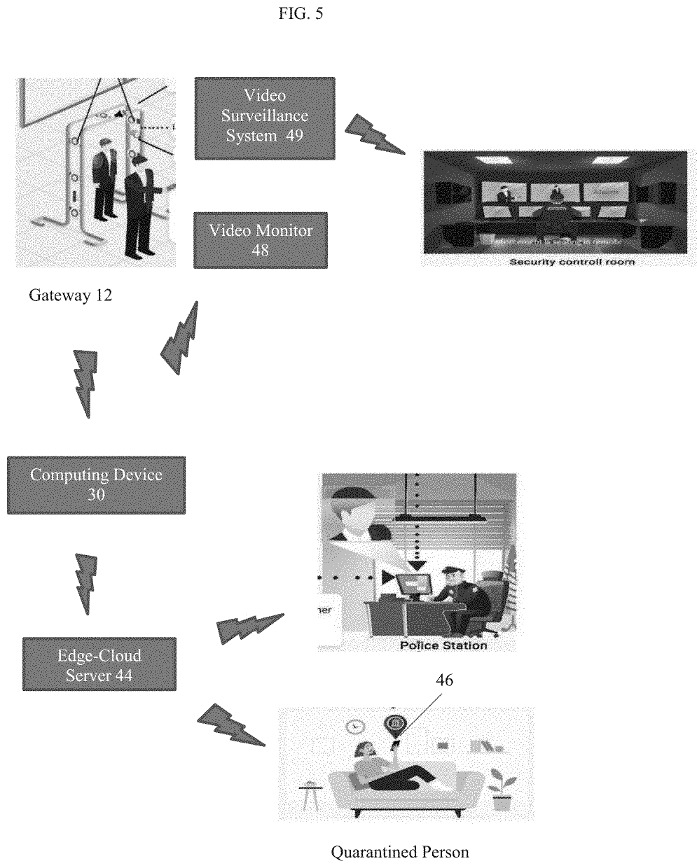

The individual with the elevated body temperature next is instructed to download onto his or her smart phone 46, or other mobile computing device, an application 47 that facilitates registration of the individual (activity 110). The instructions can be provided to the individual via a video monitor 48 shown in FIG. 5, physical signage, and/or a human attendant located proximate the exit of the gateway 12. The edge-cloud server 44 can be equipped with facial recognition software that, based on the image obtained by the video camera 16 and transmitted to the edge-cloud server 44, determines whether the individual previously has been registered. In such cases, the edge-cloud server 44 can notify the individual immediately, via SMS text, e-mail, or other suitable means, of the requirement to update his or her registration information.

The individual with the elevated body temperature is instructed to complete the registration process, or the updated registration process, before leaving the area proximate the exit of the gateway 12 (activity 112). An individual not in possession of a smart phone 46 can be directed to a nearby security desk or other location at which the individual can be registered without the use of a smart phone 46.

The individual with the elevated body temperature can be monitored upon exiting the gateway 12, to help ensure that that the individual completes the registration process (activity 114). The monitoring can be performed by security personnel stationed at the gateway 12; a video surveillance system 49 monitored from a centralized security room as depicted in FIG. 5; or by other suitable means. In applications where a manned security presence is maintained at the gateway 12, the security personnel or local law-enforcement official can enforce registration of the individual on site, within the parameters of the applicable laws and or emergency regulations.

In situations where total automation of the screening process is desired, e.g., if an on-site security presence is too expensive to maintain or otherwise is unavailable, a fully automated video surveillance system (not shown) can be installed to monitor people passing through the gateway 12. If an individual leaves the area without registering, despite the virtual or physical signage warning against doing so and the activation of the alarm 18, the video image of the individual obtained from the video camera 16 can be relayed to the local law enforcement authority. The law enforcement authority can attempt to match the image to a facial image stored in a local, regional, or national data base, using facial recognition techniques. If a match is found, the individual can be contacted and notified of his or her obligation to register self-quarantine, and appropriate enforcement action can be undertaken to ensure compliance. In addition to the image obtained from the camera 16, the images obtained from video surveillance system can be used in subsequent enforcement proceedings as evidence showing that the individual left area without registering. In applications where the system 10 is used in a private setting in which facial recognition data is readily available for most of the individuals passing through the gateway 12, such as an office building or an industrial operation, compliance based on facial recognition readily can be enforced, and generally is considered appropriate employee supervision by an employer.

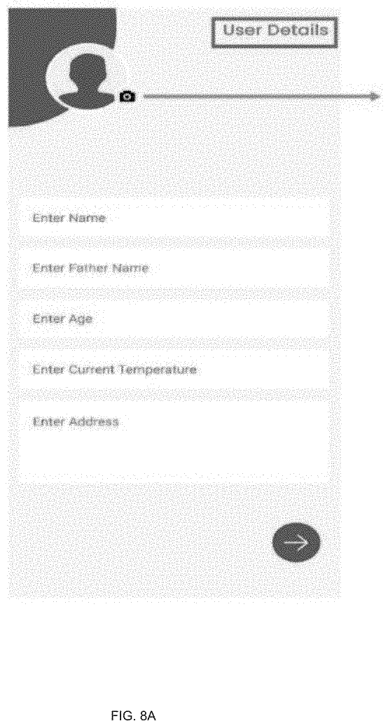

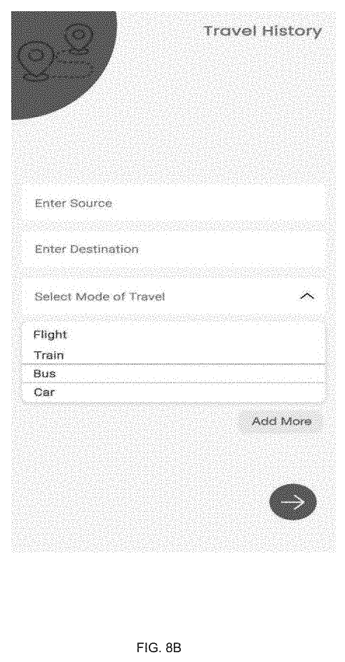

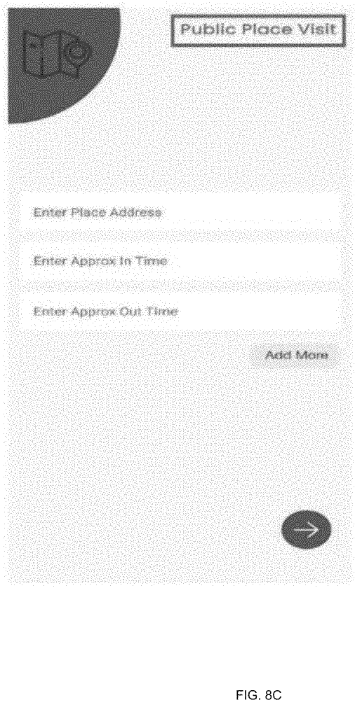

Once the individual with the elevated body temperature has downloaded the application 47, the individual is prompted to register (activity 110). The registration process can be coordinated by the edge-cloud server 44, which communicates with the smart phone 46 via a cellular network, Wi-Fi, or other suitable means. FIGS. 8A-8C depict examples of interactive registration pages that can be displayed on the individual's smart phone 46 to guide the individual through the registration process. After the individual enters his or her name, the individual is prompted to enter, for example and without limitation, the following information: the address to which the individual is proceeding, the planned length of stay at that location, contact information such as a phone number, the individual's age, etc. (activity 112).

Referring to FIG. 8B, the individual next is prompted to enter his or her recent travel history including, without limitation, the specific places traveled to and from, the length of stay, the mode of travel, and the identities of other people with whom the individual traveled or otherwise had close and extended contact, etc. After entering the recent travel history, the individual is further prompted to enter the identifies and locations of public places that the individual recently has visited, along with the approximate arrival and departure times to and from those places, as shown in FIG. 8C (activity 112).

The individual with the elevated temperature then is instructed, via a display on the smart phone 46 and/or a notification sent via SMS text, e-mail, registered mail, and/or other suitable means, that the individual is required to self-quarantine at his or her destination for a predetermined period of time, or until a licensed medical professional has indicated that it is no longer necessary to quarantine (activity 116). The quarantine period can be determined by the local health authority or other body responsible for public health it the local area. Detailed quarantine instructions can be sent via SMS text, e-mail, registered mail, and/or other suitable means.

One the individual has registered, the edge-cloud server 44 transmits the registration information, including the individual's identity and image; and the event count to a local or central governmental authority, such as a local police station as shown in FIG. 5 (activity 118). In this particular example, the police station maintains data regarding individuals identified by the system 10 as having elevated temperatures, and who are present within the geographic area served by that particular police station. For example, in the time frame depicted in FIGS. 6 and 7, six events had occurred, and the registration process for five of the six cases had been completed.

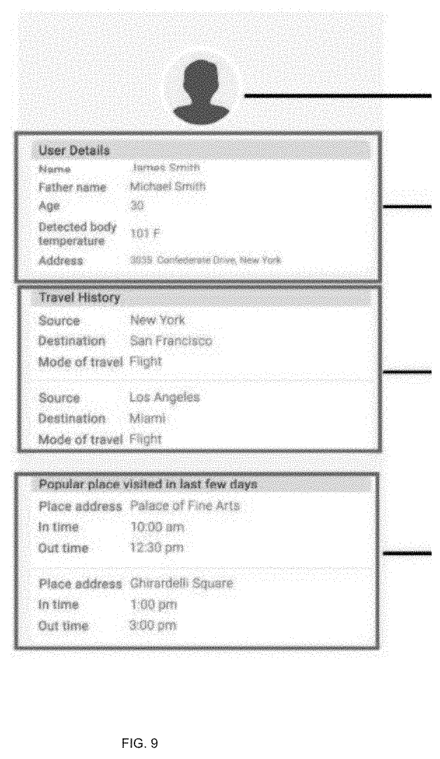

In addition to notifying the local police station for the purpose of monitoring and enforcing the self-quarantine, some or all of the registration information can be sent to one or more governmental authorities or non-governmental organizations having an interest tracking individuals exhibiting symptoms of an infectious disease (activity 120). For example, the registration information, including the location and travel history of the individual, can be sent to a centralized governmental health agency for use in compiling statistics regarding the presence and spread of a particular disease currently of interest. FIG. 9 depicts an example of a format for e-mails that can be used to notify public health officials of persons identified as potentially having an infectious disease of interest. Specifically, the individual's photo is provided along with information identifying the individual, the individual's travel history, and public places visited by the individual during the most recent several days. This information can be combined with similar information for other individuals also suspected of having the disease. The information thus can form a basis for health officials to take appropriate action to control the spread of the disease, and can provide governments with a tool to help manage pandemics and other health crises resulting from the spread of an infectious disease.

As further illustrated in FIG. 5, following the registration process, the police or other enforcement authority is informed of whether the person registered is remaining at the address provided during the registration process (activity 120). In the event the person is not confirmed to be at that address, appropriate action can be taken by the police or other governmental authorities to locate the individual, and have the individual return to his or her residence for the remainder of the quarantine period.

The system 10 is configured to track and monitor the registered individual during the quarantine period, and to inform the appropriate governmental authority when the individual is not located at the registered address, i.e., at the location at which the individual is expected to self-quarantine (activity 122). In particular, the edge-cloud server 44 can be configured with software that permits the edge-cloud server 44 to perform a geofencing process to determine whether the registered individual is remaining at the registered address. For example, the application 47 can cause the smart phone 46 to transmit location data to the edge-cloud server 44. The location data can be generated using the off-the-shelf capabilities of the smart phone 46. The edge-cloud server 44 is configured to continually track the location of the smart phone 44 associated with the registered individual, and to determine whether the smart phone 44, and presumably the individual, remain within the registered location. The location tracking can be performed on a non-continuous basis, e.g., once every hour, in alternative embodiments.

When the edge-cloud server 44 detects that the smart-phone 46 is not located at the registered quarantine location, the edge-cloud server 44 immediately sends an electronic notification to the registered individual (activity 124). The notification can be sent as an SMS text, an e-mail, or in another suitable form, and can instruct the individual to return to the registered quarantine location for the duration of the quarantine period. In addition, an electronic notification also can be sent immediately to the police station or other governmental authority, stating the monitored individual is not present at the registered quarantine location (activity 126). The police or other governmental authority can take appropriate enforcement action within the bounds of the local laws and emergency regulations. For example, the authorities may issue a warning by phone, SMS text, e-mail, etc., instructing the individual to return to the registered quarantine location or face fines or other enforcement action.

The edge-cloud server 44 maintains a record of when the registered individual is, and is not present at the registered quarantine location. This information can be used by the police or other governmental authority to identify chronic non-compliance with the self-quarantine requirement, which in turn can be used to determine the appropriate level enforcement action.

In alternative embodiments, the location of the registered individual can be tracked using a means other than the smart phone 46. For example, the registered individual can be required to wear an ankle bracelet or other suitable tracking device that is secured to the individual and transmits the individual's location to the edge-cloud server 44.

The system 10, method 100, and variants thereof, provide thermal imaging of people in a public area, and facilitate the self-imposition of a quarantine if the thermal image reveals an elevated body temperature possibly indicating that the person of interest is suffering from a particular disease.

The ability to detect and isolate individuals suspected of having an infectious disease can significantly reduce the spread of the disease, and can be particularly beneficial during an epidemic or pandemic. The application-based registration process helps government officials to track whether registered individuals have self-quarantined as required, allowing enforcement action to be taken if necessary to enforce compliance. The system 10 can be implemented at relatively low cost and in significant numbers, and thus and can facilitate widespread screening across a population. Also, the use of a gateway, such as the gateway 12, at the entrance or exit of a particular area helps to ensure that all of the individuals entering or exiting the area are subjected to the screening process.

Due to the use of edge-cloud computing and internet-of-things (IoT) technology, the system 10, method 100, and variants thereof can provide immediate notification of a possible infectious disease to the individual with the elevated temperature, and to national, regional, and local health authorities and organization. The collection of historical data by the system 10, and the forwarding of such data to appropriate public health authorities or organizations allows such authorities and organizations to track the number of people suspected of having an infectious disease, and to predict, monitor, and better control the spread of the disease.

The system 10, and variants thereof, possess a high degree of automation, automatically reporting the detection of high temperatures in screened individuals; automatically monitoring whether such individuals are self-quarantining; and automatically detecting when an individual is not complying with the self-quarantine requirement. The system 10, and variants thereof, are equipped with discrete event detection capabilities, and can perform location-based tracking of individuals having body temperature indicating that the individual may be infected with an disease currently of particular interest. Also, the system 10 and its variants can report non-compliance with the self-quarantine requirement automatically to law enforcement authorities, thereby helping to increase compliance with the requirement.

The system 10, method 100, and variants thereof can be used, for example, by national, state, and county government officials who wish to track the risk of infection by determining the percentage of their population who have a fever and are self-quarantining. Private companies, in both industrial and office settings, can use the system 10, method 100 and variants thereof to help ensure that employees and visitors with an elevated temperature, and possibly suffering from an infection disease, cannot enter into the workplace. The system 10, method 100 and their variants also can be used in passenger hubs, such as bus and train terminals, to help avoid the spread of infectious diseases among passengers and staff.

As disclosed above and from the foregoing description of exemplary embodiments of the invention, it will be readily apparent to those skilled in the art to which the invention pertains that the principles and particularly the compositions and methods disclosed herein can be used for applications other than those specifically mentioned. Further, as one of skill in the art will readily appreciate from the disclosure of the invention as set forth hereinabove, apparatus, methods, and steps presently existing or later developed, which perform substantially the same function or achieve substantially the same result as the corresponding embodiments described and disclosed hereinabove, may be utilized according to the description of the invention and the claims appended hereto. Accordingly, the appended claims are intended to include within their scope such apparatus, methods, and processes that provide the same result or which are, as a matter of law, embraced by the doctrine of the equivalents respecting the claims of this application.

We claim:

1. A system for identifying and isolating persons suspected of having an infectious disease, comprising:
a gateway configured to define a space for the passage of a person therethrough;
a temperature sensor configured to, during operation, generate a temperature reading of the person as the person passes through the space; and
a computing system communicatively coupled to the temperature sensor, wherein the computing system is configured to, during operation, determine whether the temperature reading exceeds a limit, and if so, prompt the person to input information into the computing system.

2. The system of claim 1, further comprising a video camera communicatively coupled to the computing system and configured to, during operation, generate an image of the person as the person passes through the space.

3. The system of claim 2, further comprising an alarm communicatively coupled to the computing system and configured to, during operation, generate a visual and/or an audible alert;
wherein the computing device is further configured to, during operation, activate the alarm when the temperature reading exceeds the limit.

4. The system of claim 3, wherein at least one of the temperature sensor, the video camera, and the alarm are mounted on the gateway.

5. The system of claim 1, wherein the temperature sensor is an infrared sensor.

6. The system of claim 1, wherein the information comprises registration information relating to at least one of: an identify of the person; a residence of the person; a travel history of the person during a predetermined time period; and a listing of public places visited by the person during a predetermined time period.

7. The system of claim 1, wherein the computing system is further configured to, during operation, monitor a location of the person.

8. The system of claim 7, wherein the computing system is further configured to, during operation, determine whether the person is located at a specific location.

9. The system of claim 8, wherein the computing system is further configured to, during operation, generate and send a notification upon determining that the person is not located at the specific location.

10. The system of claim 8, wherein the specific location is a residence of the person.

11. The system of claim 9, wherein the computing system is further configured to, during operation, send the notification to the person and/or to a governmental authority.

12. The system of claim 1, wherein the computing system is further configured to, during operation, prompt the person to input the information into the computing system by way of a smart phone.

13. The system of claim 8, wherein the computing system is further configured to, during operation, determine whether the person is located at the specific location based on location information generated by a smart phone.

14. The system of claim 1, further comprising an ultrasonic sensor communicatively coupled to the computing device and configured to, during operation, generate an output representing a distance between the ultrasonic sensor and the person as the person passes through the space.

15. The system of claim 14, wherein the computing system is further configured to, during operation, determine a location of the person in relation to the gateway based on the output of the ultrasonic sensor; and, if the location lies outside of a particular range, to ignore the temperature reading.

16. The system of claim 1, wherein the computing system is further configured to, during operation, calculate a core body temperature of the person based on the temperature reading, an ambient temperature measured at or near the gateway, and a relationship between ambient temperature, skin temperature, and core body temperature.

17. The system of claim 16, wherein the computing system is further configured to, during operation, determine whether the core body temperature exceeds the limit, and if so, prompt the person to input the information into the computing system.

18. The system of claim 16, wherein the computing system is further configured to, during operation, interpret a core body temperature that exceeds the limit as an indication that the person has a fever.

19. The system of claim 16, wherein the relationship between ambient temperature, skin temperature, and core body temperature accounts for differences between skin temperature and core body temperature based on the ambient temperature.

20. The system of claim 16, wherein the relationship between ambient temperature, skin temperature, and core body temperature is determined using a training data set.

21. A process for identifying and isolating persons suspected of having an infectious disease, comprising:
directing a person through a space;
measuring a temperature of the person as the person passes through the space;
determining whether the temperature exceeds a limit, and if so, prompting the person to provide information regarding the identity and residence of the person; and
monitoring a location of the person.

22. The process of claim 21, wherein directing a person through a space comprises directing the person through a gateway that defines the space.

23. The process of claim 21, further comprising obtaining a visual image of the person as the person passes through the space.

24. The process of claim 23, further comprising generating a visual and/or an audible alert when the temperature reading exceeds the limit.

25. The process of claim 21, further comprising generating an alert if the temperature reading exceeds the limit.

26. The process of claim 21, further comprising monitoring a location of the person.

27. The process of claim 21, further comprising determining whether the person is located at a specific location.

28. The process of claim 27, further comprising generating and sending a notification upon determining that the person is not located at the specific location.

29. The process of claim 27, wherein determining whether the person is located at a specific location comprises determining whether the person is located at a residence of the person.

30. The process of claim 21, further comprising sending the notification to the person and/or to a governmental authority.

31. The process of claim 21, further comprising prompting the person to input information into the computing system by way of a smart phone.

32. The process of claim 27, further comprising determining whether the person is located at the specific location based on location information generated by a smart phone.

33. The process of claim 21, further comprising determining a position of the person within the space; and, if the position of the person lies outside of a particular range, ignoring the measured temperature.

34. The process of claim 33, wherein determining a position of the person within the space comprises determining the position of the person using an ultrasonic sensor.

35. The process of claim 21, further comprising calculating a core body temperature of the person based on the measured temperature of the person, an ambient temperature measured at or near the gateway, and a relationship between ambient temperature, skin temperature, and core body temperature.

36. The process of claim 35, further comprising determining whether the core body temperature exceeds the limit, and if so, prompting the person to provide the information regarding the identity and residence of the person.
